(19) United States
(12) Patent Application Publication
(10) Pub. No.: US 2011/0172592 A1
(43) Pub. Date: Jul. 14, 2011

(54) MEMBRANE PORT COMPATIBLE WITH STEAM STERILIZATION

(75) Inventor: Patrick Jong Lee, Long Grove, IL (US)

(73) Assignees: BAXTER INTERNATIONAL INC., Deerfield, IL (US); BAXTER HEALTHCARE S.A., Glattpark (Opfikon) (CH)

(21) Appl. No.: 12/685,399

(22) Filed: Jan. 11, 2010

(51) Int. Cl. A61M 39/16; A61M 1/28

(52) U.S. Cl. 604/29

(57) ABSTRACT

A medical connector comprising a body having a first end, a second end and a body wall defining an interior. A removable cap is fixed to the first end to seal the first end, the cap having a first filter. A filter plug having a proximal end, a distal end, a second filter and defining a spike path on an interior of the filter plug fluidly seals to an inside surface of the body wall. The distal end of the filter plug also has a removable membrane that blocks the spike path. The medical connector also provides a steam path with at least a portion of which is provided between the body wall and the filter plug. The medical connector can also comprise a first port and second port connected together to define the connector interior. The medical connector can provide the first filter on the connector body.

### BACKGROUND

The present disclosure relates to an apparatus for sterile medical fluid delivering and in particular to the delivering of a dialysis solution.

Due to disease or other causes, a person's renal system can fail. In renal failure of any cause, there are several physiological derangements. The balance of water, minerals and the excretion of daily metabolic load is no longer possible in renal failure. During renal failure, toxic end products of nitrogen metabolism (urea, creatinine, uric acid, and others) can accumulate in blood and tissues.

Kidney failure and reduced kidney function have been treated with dialysis. Dialysis removes waste, toxins and excess water from the body that would otherwise have been removed by normal functioning kidneys. Dialysis treatment for replacement of kidney functions is critical to many people because the treatment is life saving. One who has failed kidneys could not continue to live without replacing at least the filtration functions of the kidneys.

One type of dialysis is peritoneal dialysis. Peritoneal dialysis uses a dialysis solution or “dialysate”, which is infused into a patient's peritoneal cavity through a catheter implanted in the cavity. The dialysate contacts the patient's peritoneal membrane in the peritoneal cavity. Waste, toxins and excess water pass from the patient's bloodstream through the peritoneal membrane and into the dialysate. The transfer of waste, toxins, and water from the bloodstream into the dialysate occurs due to diffusion and osmosis, i.e., an osmotic gradient occurs across the membrane. The spent dialysate drains from the patient's peritoneal cavity and removes the waste, toxins and excess water from the patient. This cycle is repeated.

There are various types of peritoneal dialysis therapies, including continuous ambulatory peritoneal dialysis (“CAPD”) and automated peritoneal dialysis (“APD”). CAPD is a manual dialysis treatment, in which the patient connects an implanted catheter to a drain and allows a spent dialysate fluid to drain from the patient's peritoneal cavity. The patient then connects the catheter to a bag of fresh dialysate and manually infuses fresh dialysate through the catheter and into the patient's peritoneal cavity. The patient disconnects the catheter from the fresh dialysate bag and allows the dialysate to dwell within the cavity to transfer waste, toxins and excess water from the patient's bloodstream to the dialysate solution. After a dwell period, the patient repeats the manual dialysis procedure.

In CAPD the patient performs several drain, fill, and dwell cycles during the day, for example, about four times per day. Each treatment cycle typically takes about four hours. APD is similar to CAPD in that the dialysis treatment includes a drain, fill, and dwell cycle. APD machines, however, perform three to four cycles of peritoneal dialysis treatment automatically, typically overnight while the patient sleeps. Like CAPD, APD machines connect fluidly to an implanted catheter, to one or more sources or bags of fresh dialysate and to a fluid drain.

The APD machines pump fresh dialysate from the dialysate source, through the catheter, into the patient's peritoneal cavity and allow the dialysate to dwell within the cavity so that the transfer of waste, toxins and excess water from the patient's bloodstream to the dialysate solution can take place. The APD machines then pump spent dialysate from the peritoneal cavity, though the catheter, to the drain. APD machines are typically computer controlled so that the dialysis treatment occurs automatically when the patient is connected to the dialysis machine, for example, when the patient sleeps. That is, the APD systems automatically and sequentially pump fluid into the peritoneal cavity, allow for a dwell, pump fluid out of the peritoneal cavity and repeat the procedure. As with the manual process, several drain, fill, and dwell cycles will occur during APD. A “last fill” is typically used at the end of APD, which remains in the peritoneal cavity of the patient when the patient disconnects from the dialysis machine for the day.

Performance of dialysis, however, is subject to certain considerations. For example, it is important to maintain sterility between the dialysate source and the patient. Failure to maintain sterility may allow germs or pathogens to reach the patient's peritoneum and cause peritonitis. Peritonitis can cause the patient to feel extreme pain and if not treated properly can result in death. Different methods of sterilization are available, such as steam sterilization. Steam sterilization is desirable because it avoids the drawbacks of other methods, such as requiring a chemical application or the yellowing and odor associated with radiation sterilization.

It is also important to provide a dialysis delivery system that is easy to use, particularly for APD systems in which multiple dialysate sources are connected to an APD machine. Here, it is beneficial to provide dialysate solution containers with pre-attached tubing so that a patient can connect the pre-attached tubing for each container to the APD machine instead of having to manipulate each dialysate container. During connection, it is also important to minimize or eliminate the risk of fluid leaking when connecting to the APD machine. Also, when using a multiple chamber dialysate bag, which requires a full mixing of the components of the chambers prior to patient delivery, it is important to provide dry tubing pre-attached to the bag that stays dry until the mixed fluid components are ready for patient delivery. That is, if one of the component fluids is allowed to flood the line before the components are mixed, that slug of fluid will not be mixed properly prior to being delivered to, e.g., a patient.

A need therefore exists for a tubing connector that allows for steam sterilization of a dry tube and for connecting a pre-attached dry tube and corresponding dialysate bag to a dialysis machine.

### SUMMARY

The present disclosure sets forth a medical connector apparatus that facilitates fluid communication between a dialysate bag and a disposable cassette. The medical connector is configured to allow steam sterilization of its interior and a connected tube and to maintain the sterilized state for connection to a spike on the disposable cassette. The medical connector uses a cap for enclosing the connector. The connector in one embodiment is a female connector that becomes spiked, on one end, by the spike of a disposable cassette. The connector connects, on its other end, to a tube that is attached to a medical bag.

In one embodiment, a body of the medical connector has a first end, a second end and a body wall defining an interior. The cap is fixed to the first end to seal the first end, and includes a first filter designed to allow steam to enter the connector for sterilization purposes while preventing contaminants from entering. The body also includes a filter plug fluidly sealed to the wall of the body. The filter plug is hollow and has an open proximal end and a distal end closed by a membrane. The filter plug also has a second filter, like the first filter, which allows for steam sterilization of the connector interior. The interior of the hollow plug defines a spike path for a spike, e.g., cassette spike, to enter the connector first end and pierce the membrane to facilitate fluid communication with a component connected to the connector second end, e.g., a dialysate bag or a tube pre-attached to the dialysate bag.

The second filter can be positioned in various locations along the filter plug including, for example, at the open proximal end, outside the spike path, or along the spike path. The second filter can have various shapes and configurations including, for example, a disc shape or ring shape, and can also include a plurality of smaller filters of the same shape, e.g., a circular shape. The second filter is positioned along a steam path, which is formed to allow steam to pass through the first filter and through the connector interior. The steam path can be positioned at least partially outside or completely outside the spike path.

In a second embodiment, the medical connector has a cap with a first filter, a first port and a second port that connects to each other to define the body of the connector and a spike path through the interior of the connector. A filter member is arranged at or near the intersection of the first and second port. The filter member includes a second filter and a membrane. A generally disc-shaped second filter is attached to the second port and surrounds the membrane. The membrane is bonded to the second filter to maintain the membrane in a position blocking the spike path such that the cassette spike pierces the membrane to facilitate fluid communication. The first and second ports are also configured to be connected to define a steam path located at least partially outside the spike path.

In this second embodiment, the second filter can have different configurations. One configuration has a front face and side face that form a portion of the steam path. This configuration allows steam to pass through the front face and exit the side face, passing around the membrane and steam sterilizing the interior of the connector. Another configuration has a front face and a rear face that form the portion of the steam path. In this configuration, the second port can also include a bore adjacent the rear face so that steam can pass through the front face, pass through the rear face, and exit through the bore to pass around the membrane and steam sterilize the interior of the connector.

In either of the first and second embodiments, the first filter is arranged alternatively on an inner surface of the wall of the connector. In this alternative arrangement, the cap has no filter and the wall defines a passageway, blocked by the first filter, which allows steam to pass through the passageway, through the first filter, and into the connector interior. The steam can then pass through the second filter described above for any of the preceding embodiments.

The above apparatus enables a method of sterilization to occur in which steam passes through a cap or wall first filter to sterilize a portion of the connector, and then passes through a second filter within the connector to sterilize the remainder of the connector. It has been found that the first and second filters, which can be hydrophobic filters, allow steam to pass while preventing contaminants to pass. The filters allow for sterilization of a connector to occur after the connector is fixed to a tube that is pre-attached to a medical container, and thus allow a sterilized state to be maintained before connection with a disposable cassette.

It is accordingly an advantage of the present disclosure to provide an improved apparatus for steam sterilization of a tube and attached connector.

It is another advantage of the present disclosure to provide an improved connector that can be sterilized (at least substantially) along with a tube attached to the connector, while keeping the tube and connector sealed from exterior contaminants.

It is a further advantage of the present disclosure to provide an improved system for at least substantially sterilizing a connector fixed to a tube that is pre-attached to a dialysate bag.

Additional features and advantages are described herein, and will be apparent from the following Detailed Description and the figures.

### BRIEF DESCRIPTION OF THE FIGURES

FIG. 1 is a perspective view of a dialysate bag having pre-attached dry tubing capped by a connector of the present disclosure.

FIGS. 2A to 2C are cross-sectional views of one embodiment of a plug-type connector of the present disclosure having a removable cap for connecting to a cassette spike.

FIG. 3 is a cross-sectional view illustrating another embodiment of a plug-type connector of the present disclosure.

FIG. 4 is a cross-sectional view illustrating yet another embodiment of a plug-type connector of the present disclosure.

FIG. 5 is a top view of one embodiment of a micronfilter employed in the capped connector of FIG. 4.

FIG. 6A is a cross-sectional view of one embodiment of a two-part connector of the present disclosure.

FIG. 6B is a cross-sectional view illustrating another embodiment of a two-part connector of the present disclosure.

FIG. 6C is a cross-sectional view illustrating an embodiment of a two-part connector having an integrated micronfilter of the present disclosure.

### DETAILED DESCRIPTION

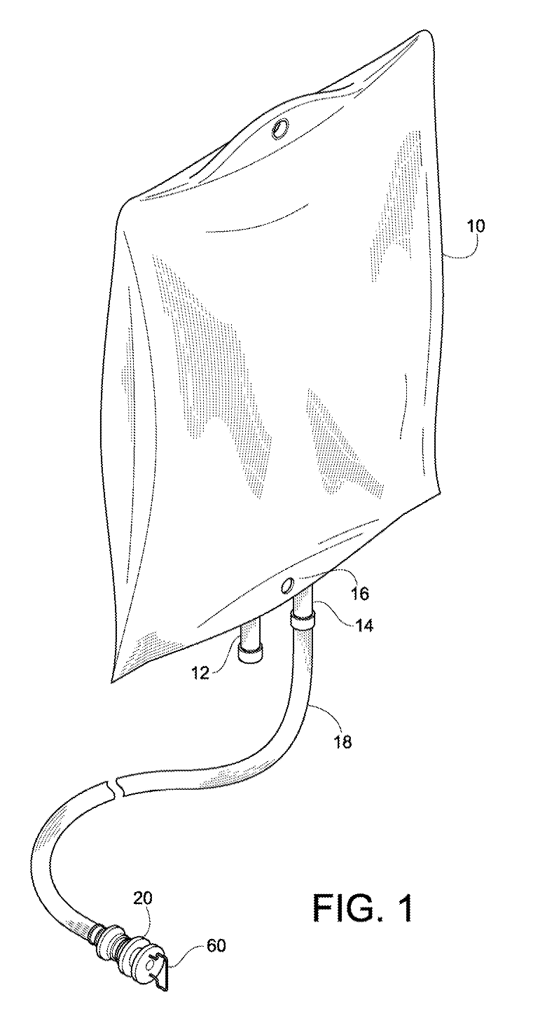

Referring now to the drawings and in particular FIG. 1, a dialysate bag 10 is illustrated. Bag 10 includes an inlet 12, an outlet 14 and a seal 16. Seal 16 is a frangible seal such as a moon seal or peel seal that seals the contents of bag 10 within the bag. One end of a tube 18 is pre-attached to outlet 14. A port 20 is attached to the distal end of tube 18. Port 20 includes a cap 60 enclosing a connector end of port 20 to seal the interior of port 20. Port 20 and cap 60, as discussed in detail below, seal tube 18 to prevent contamination of the interior of tube 18 while also allowing for steam sterilization of the interior of tube 18 and port 20 without removal of cap 60. As used herein, the terms “sterilize,” “sterilizing” and “sterilization” refer to a medically acceptable level of sterilization, which may be highlighted in certain places by the phrase “at least substantially.”

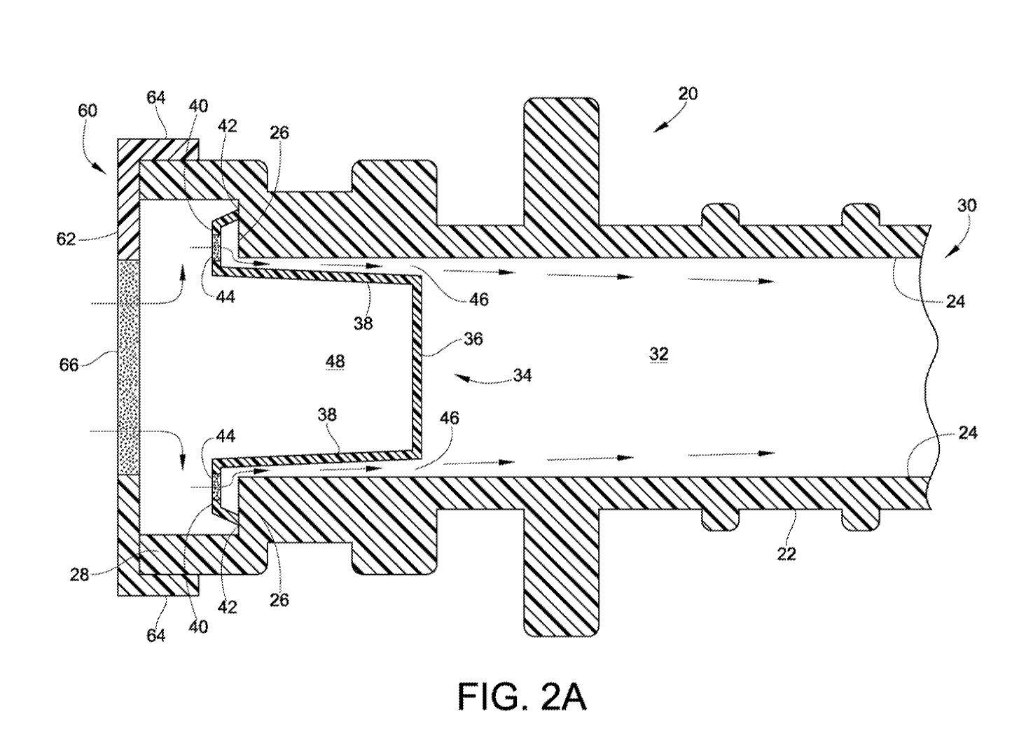

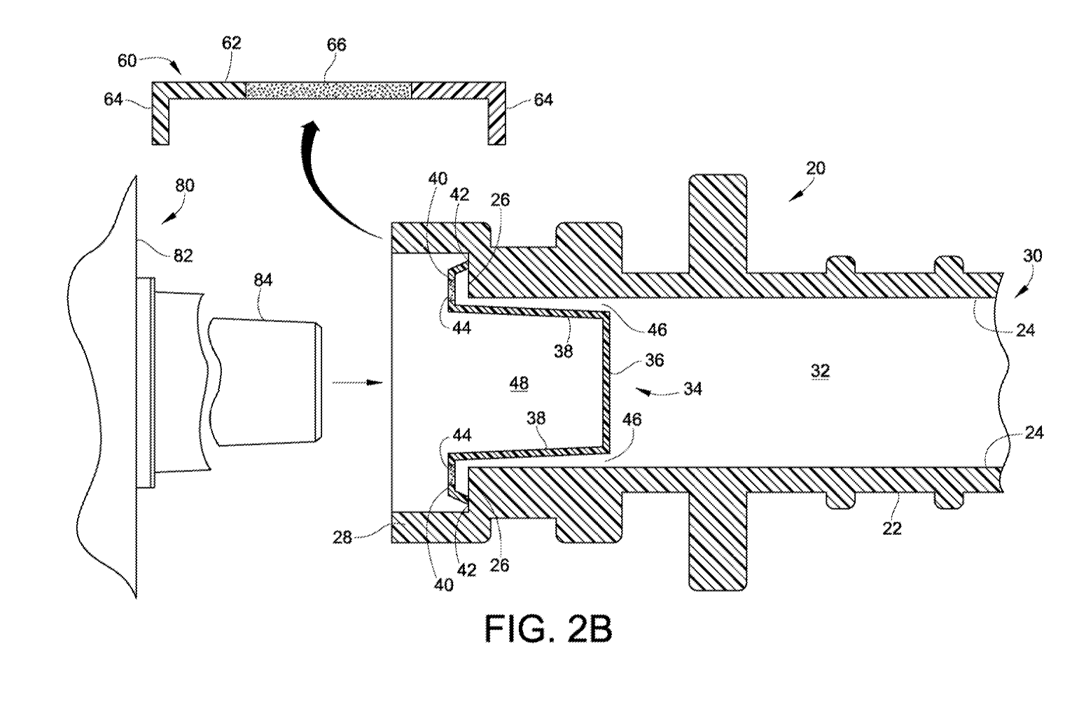

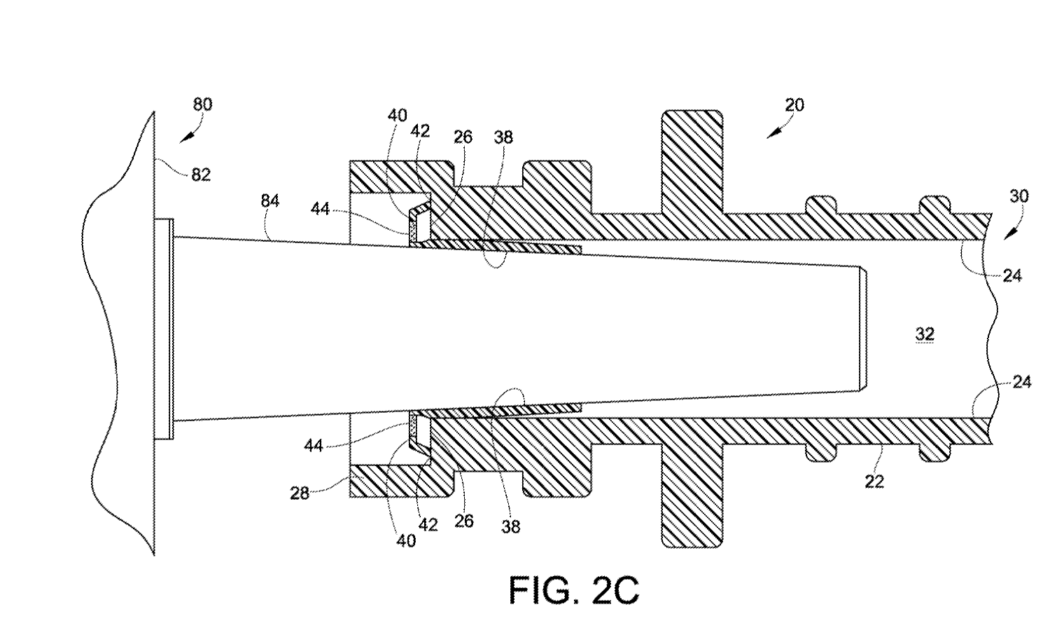

FIG. 2A illustrates one embodiment of port 20 and cap 60 connected to the port. Port 20 is illustrated as having a body 22, which can be made of various semi-rigid materials including, for example, polyethylene (“PE”), polypropylene (“PP”), styrene-b-(ethylene-co-butylene)-b-styrene (“SEBS”), styrene butadiene rubber (“SBR”), hydrogenated styrene butadiene rubber (“HSBR”), sulfonated styrene butadiene rubber (“SSBR”), polybutadiene (“PB”), and combinations thereof. Body 22 forms a wall 24, a first port end 28 that is engaged by a cassette spike 84 as shown in FIGS. 2B and 2C, and a second port end 30 opposite first port end 28 for connecting sealingly to tube 18. Body 22 also includes a step portion 26, adjacent wall 24, formed at first port end 28. Step portion 26 aids in receiving cassette spike 84.

Within port 20 is a filter plug 34 configured to seal off an interior 32 of port 20 from contaminants, while allowing for steam sterilization of the interior 32 as well as the interior of tube 18. Filter plug 34, as illustrated in FIG. 2A has a “top-hat” configuration in which filter plug 34 includes a plug face (membrane) 36 spaced inwardly from first port end 28 and extending laterally across interior 32. Plug face 36, as discussed below, serves as a membrane that is pierced by cassette spike 84 to allow fluid communication between a disposable pumping cassette and a medical bag attached to tube 18. A cylindrical plug wall 38 extends towards first port end 28 from membrane 36. Plug wall 38 may extend parallel to interior wall 24 or may taper outwards towards wall 24 as plug wall 38 extends towards first port end 28. Plug flange 40, which extends laterally outward from the end of plug wall 38 opposite membrane 36, is attached to step portion 26 of port body 22 at a bonding portion 42 of filter plug 34. Bonding portion 42 can be composed of polyethylene (“PE”), PP, or a combination of the two, such that it can be heat bonded to step portion 26. Other bonding techniques may be employed, such as ultrasonic welding or solvent bonding.

Filter plug 34 and membrane 36 can be formed from a single molding or material, or can be formed from different materials that are bonded together. Filter plug 34 can be made of the same or similar material as body 22, which aids the bonding between filter plug 34 and body 22. Filter plug 34 can be made of a material, such as, PP, SEBS, SBR, HSBR, SSBR, PB, and combinations thereof.

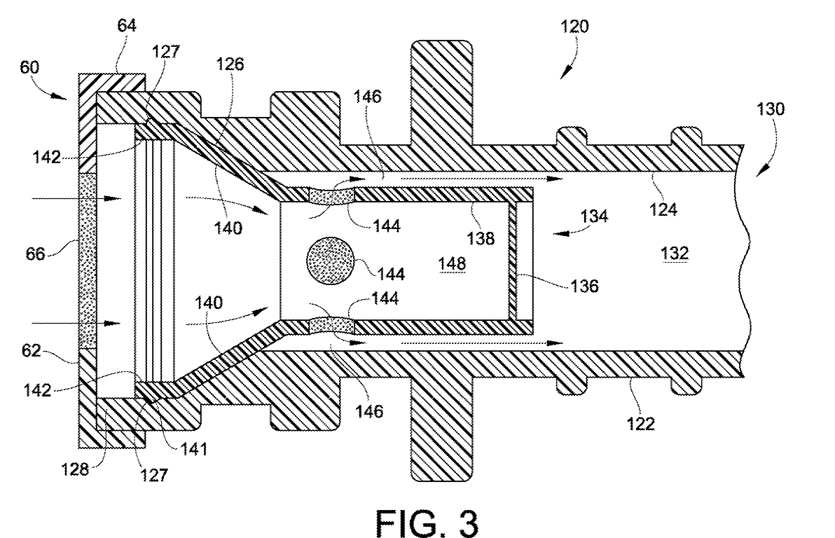

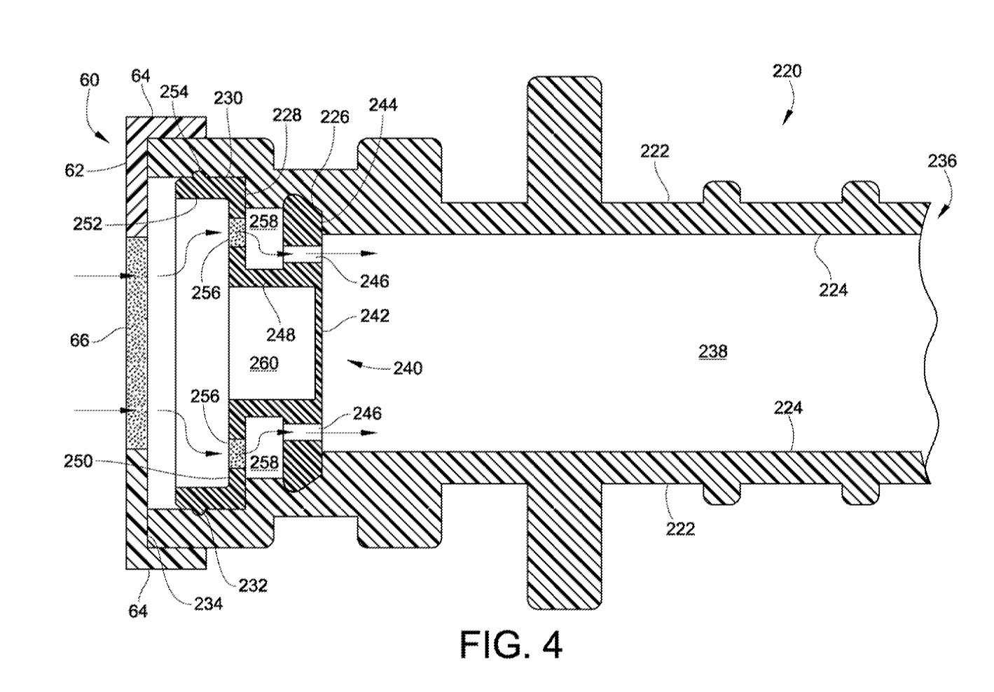

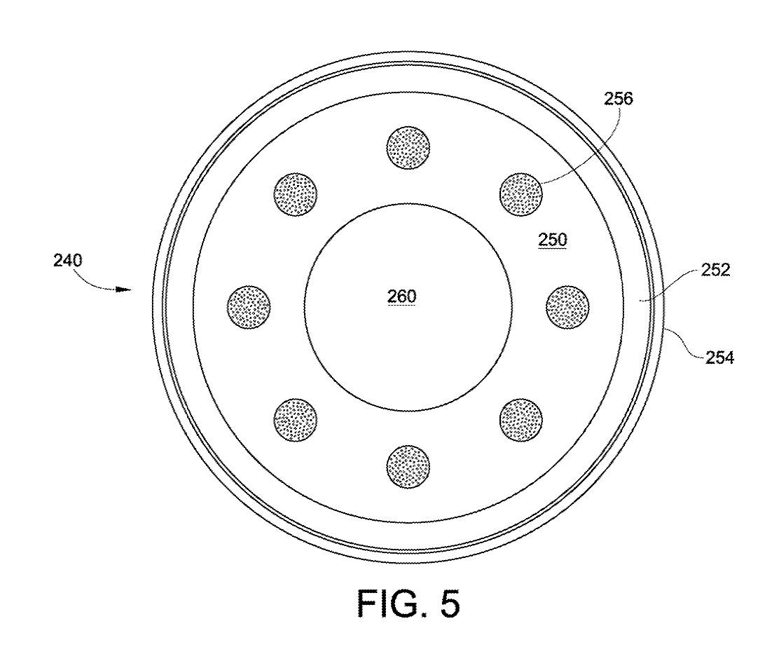

A first micronfilter 44 is incorporated into plug flange 40, which seals interior 32 of port 20 from contaminants while providing a sufficient opening for steam to pass through filter plug 34 to sterilize portions of interior 32 and tube 18. First micronfilter 44 may be disc-shaped (see FIG. 2A) or ring shaped (see FIG. 3), forming an uninterrupted disc or ring within plug flange 40, or may consist of a plurality of smaller micronfilters disposed, e.g., in a circular pattern, about plug flange 40. The smaller micronfilters may have any shape including, for example, circular micronfilters, such as those illustrated in FIG. 5 with reference to the port embodiment of FIG. 4.

The micronfilters of the present disclosure are filters with minute pore sizes of up to about 0.50 μm. The micronfilters are made from a polymer such as, for example, polyethylene (“PE”), polytetrafluoroethylene (“PTFE”), perfluoroalkoxy polymer resin (“PFA”), fluorinated ethylene-propylene (“FEP), and the like. PTFE-based micronfilters, for example, have broad chemical compatibility and have numerous applications such as clarification of acids, bases and solvents; air monitoring; filtering or venting gases, and UV spectroscopy. PTFE micronfilters are also compatible with autoclave sterilization. In addition, the micronfilter can be bonded to (e.g. radio frequency bonded [“RF bonded”] or solvent bonded), insert molded to port body 22, or laminated with a high-density polyethylene (“HDPE”) support for easier handling and for assisting in heat bonding the micronfilter to port body 22. One example of a suitable microfilter 44 or microfilter material is a 0.22 micron pore size PTFE hydrophobic fluoropore membrane, having a 175 micron thickness, produced by Millipore (part number FGLP01300).

As mentioned above, port 20 of FIG. 2A is fitted with a cap 60 at first port end 28 of port 20 to seal interior 32 of port 20 from the exterior environment. Cap 60 includes a cap face 62 that extends across first port end 28 and a cap flange 64 extending from cap face 62 to seal port 20. Cap 60 also includes a side flange 64 that wraps around first port end 28 to secure cap 60 to port 20. A second micronfilter 66, which serves the same purpose as first micronfilter 44, is incorporated into cap 60 and is made of one of the materials described above for first micronfilter 44.

Cap 60 may be silicon or silicon-based to provide a more flexible structure to ease removal of cap 60 from port 20 and promote a good seal between cap flange 64 and first port end 28. Cap 60 may also be an elastomeric medical grade plastic including, for example, polyisoprene or santoprene. Cap 60 should have a different material composition than body 22 to assist in maintaining a proper seal with first port end 28. For example, if body 22 is made of a rigid or semi-rigid material, cap 60 can be made of a semi-rigid or semi-flexible material, respectively. The internal diameter of flange 64 is also sized to press-fit or fit snugly over the outside diameter of port end 28.

To sterilize tube 18 and interior 32 of port 20, steam is injected through second micronfilter 66 of cap 60 into first port end 28. Filter plug 34 prevents steam, and any other fluids or gases, from passing through filter plug 34 into interior 32. However, first micronfilter 44 provided on flange 40 of filter plug 34 allows steam to pass through filter plug 34 and into interior 32 by way of a steam path 46 defined between plug wall 38 and port wall 24, thereby allowing steam to sterilize both port interior 32 and tube 18. Sterile port 20 is thereafter ready for connection to a dialysis cassette.

To attach port 20 and associated tube 18 to a cassette 80 to provide a dialysate path through tube 18 into cassette 80, cap 60 is removed to open first port end 28 to receive cassette spike 84, which extends from cassette body 82 as illustrated in FIG. 2B. Membrane 36 and plug wall 38 of filter plug 34 combine to provide a spike path 48, which is sized to receive cassette spike 84.

As illustrated in FIG. 2C, spike 84 pierces membrane 36 and advances through port 20, via spike path 48 illustrated in FIG. 2B. Port body 22 has a diameter such that as spike 84 extends into interior 32, spike 84 presses plug wall 38 against port wall 24, sealing off steam path 46. As a result, spike 84 and port 20 form a seal that maintains the sterility of port 20 and tube 18 and allows dialysate from bag 10 (see FIG. 1) to pass through tube 18, through port 20, through spike 84 and into cassette 80.

FIG. 3 illustrates another embodiment of a port and filter plug of the present disclosure. In this embodiment, cap 60 described above is properly sealed to a port 120. Port 120 includes a body 122 forming a wall 124 and a first port end 128 for engaging cassette spike 84 illustrated in FIGS. 2B and 2C. Body 122 includes a second port end 130 opposite first port end 128 for engaging tube 18. Body 122 also includes a tapered wall 126 adjacent wall 124 that tapers radially outwardly as it extends towards first port end 128. Body 122 also defines and annular wall groove 127 formed at first port end 128.

A filter plug 134 is fitted within port 120. Plug 134 is configured to seal interior 132 of port 120 from contaminants while allowing for steam sterilization of interior 132 and tube 18. Filter plug 134, retained within port 120, includes a membrane 136 spaced inwardly from first port end 128 and extending laterally across interior 132. A plug wall 138 extends cylindrically from membrane 136 towards first port end 128. As illustrated, wall 138 can extend at least substantially parallel with wall 124. Plug taper 140 tapers outwardly from the end of plug wall 138, opposite membrane 136, and is sized such that at least a portion of plug taper 140 fits against tapered wall 126. Filter plug 134 also includes an annular plug protrusion 141 formed at a plug end 142. Plug end 142 extends cylindrically from plug taper 140 and is sized to fit snugly within first port end 128. Plug protrusion 141 is sized and positioned to fit sealingly into wall groove 127. The combination of tapered wall 140, annular protrusion 141 and plug end 142 retain filter plug 134 in place within port 120. A first micronfilter 144 is incorporated into plug wall 138 and serves to seal interior 132 of port 120 from contaminants while providing a sufficient opening for steam to enter interior 132 and tube 18 for sterilization purposes. Micronfilter 144 can have any of the materials and configurations described above for micronfilter 44.

Membrane 136 and plug wall 138 define a spike path 148 sized to receive cassette spike 84. In a manner similar to that described above in reference to FIGS. 2B and 2C, port wall 124 is sized such that cassette spike 84 will fit snugly against port wall 124 after spike 84 punctures membrane 136 to close off first micronfilter 144 and an associated steam path 146 at least partially defined between wall 124 and plug wall 138.

To sterilize tube 18 and interior 132 of port 120, steam is injected through second micronfilter 66 of cap 60 into first port end 128. Filter plug 134 prevents steam, and any other fluids or gases, from passing through filter plug 134 into interior 132. However, first micronfilter 144 provided on plug wall 138 of filter plug 134 allows steam to pass through filter plug 134 and into interior 132 by way of steam path 146, thereby allowing the steam to sterilize both port interior 132 and tube 18. Sterile port 120 is thereafter ready for connection to cassette 80.

FIG. 4 illustrates yet another embodiment of a port and a filter plug of the present disclosure. In this embodiment, a port 220 is sealed releasably by cap 60, which has the same configuration as the cap 60 of FIG. 2A. Port 220 includes a body 222 forming a wall 224 and a first port end 234 for engaging cassette spike 84 illustrated in FIGS. 2B and 2C. Port 220 includes a second port end 236 opposite first port end 234 for engaging tube 18. Body 222 also defines an annular wall groove 226 adjacent to wall 224. Step 228 extends outward from wall groove 226 to cylindrical wall 230, which has a greater inner diameter than wall groove 226. Cylindrical wall 230 also defines an annular wall groove 232 formed adjacent to first port end 234.

Filter plug 240 is fitted within port 220 and is configured to seal the interior 238 of port 220 from contaminants, while allowing for the steam sterilization of the interior 238 and tube 18. Filter plug 240 includes a membrane 242 that is spaced inwardly from first port end 234 and that extends laterally across interior 238. A first cylindrical plug wall 248 extends parallel to port wall 224 from wall groove 226 to step portion 228. A first annular flange 244 extends radially outwardly from first cylindrical plug wall 248 near membrane 242. Annular flange 244 fits snugly into port wall groove 226. A second annular flange 250 extends from first plug wall 248 opposite from membrane 242, and fits against step portion 228 of port body 222. A second cylindrical plug wall 252 extends axially from second flange 250 and fits snugly against first wall 230 of port body 222. Second plug wall 252 has an annular plug protrusion 254 sized to fit into proximal wall groove 232. First flange 244, second flange 250, second plug wall 252 and plug protrusion 254 combine to retain sealingly filter plug 240 in place in port 220.

One or more micronfilters 256 is placed in second annular flange 250 to seal interior 238 of port 220 from contaminant, while providing a sufficient opening for steam to enter interior 238 and tube 18 for sterilization. First micronfilter 256, made of any of the micronfilter materials described herein, may be ring-shaped or disc-shaped forming an uninterrupted ring or disc within second flange 250, or may consist of a plurality of smaller micronfilters centrally disposed on second flange 250. The smaller micronfilters may have any shape including, for example, circular micronfilters 256 as illustrated in FIG. 5, which are centrally disposed on second flange 250 of filter plug 240. The interior of the plug defined by membrane 242 and first plug wall 248 provide a spike path 260 sized to receive cassette spike 84 described above in reference to FIGS. 2B and 2C.

To sterilize tube 18 and interior 238 of port 220, steam is injected through second micronfilter 66 of cap 60 into first port end 234. Filter plug 240 prevents steam, and any other fluids or gases, from passing through filter plug 240 into interior 238. However, first micronfilter 256 provided on second flange 250 of filter plug 240 allows steam to pass through filter plug 240 and into interior 238 by way of a steam chamber 258 defined between port body 222, first plug wall 248, first and second flanges 244 and 250, and a steam opening 246 formed through first flange 244. First micronfilter 256, steam chamber 258, steam opening 246, and filter plug 240 allow steam to sterilize both port interior 238 and tube 18 while sealing interior 238 from contaminants. Sterile port 220 is thereafter ready for connection to a dialysis cassette.

When cassette spike 84 punctures membrane 242, first plug wall 248 is compressed towards port body 222 such that steam opening 246 and steam chamber 258 are closed, thereby creating a fluid and gas tight seal between the cassette spike and port 220.

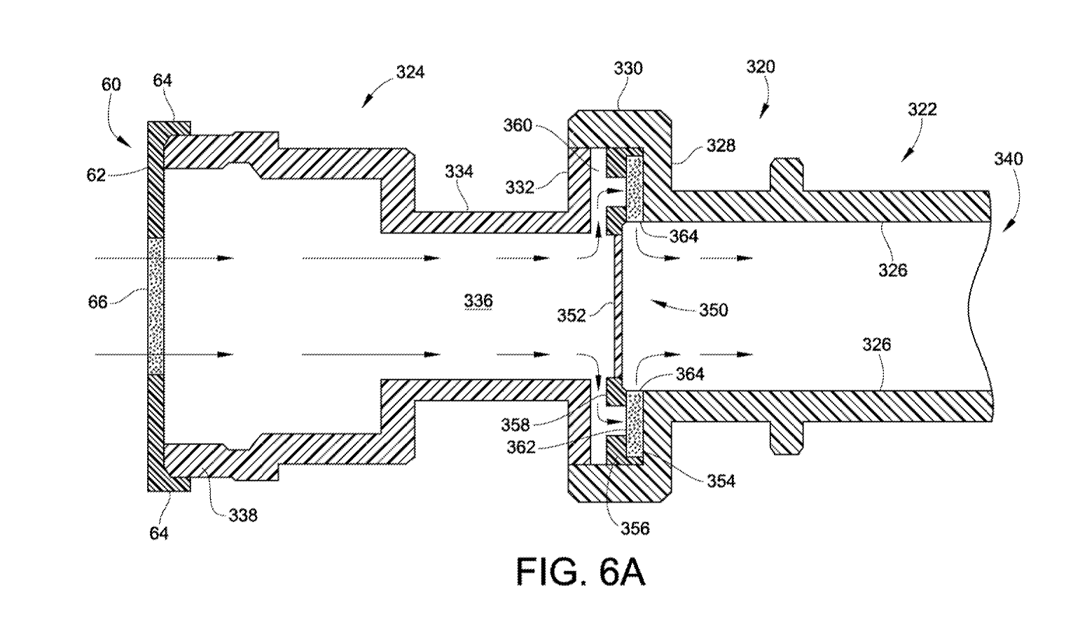

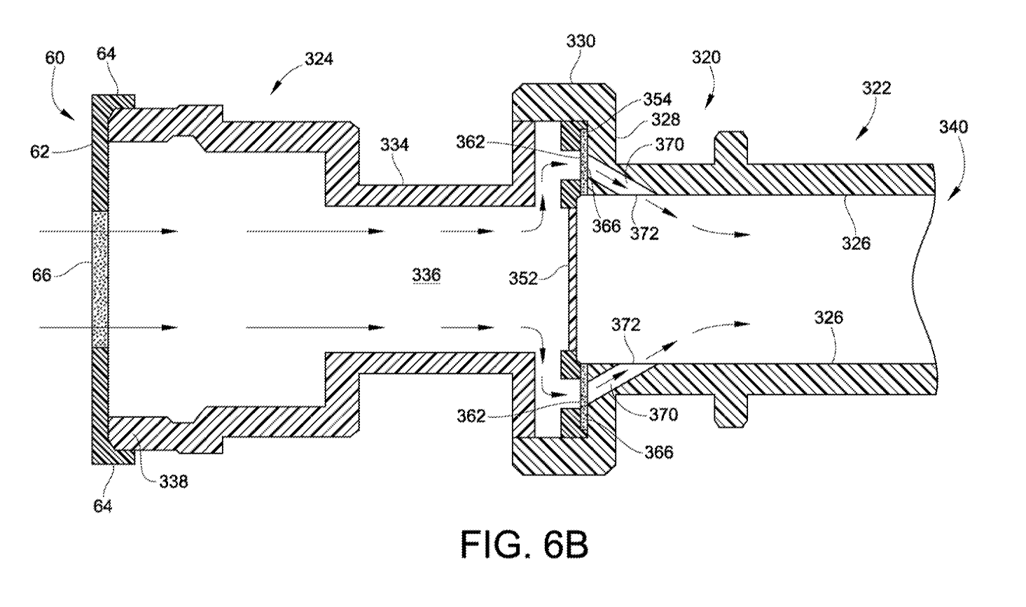

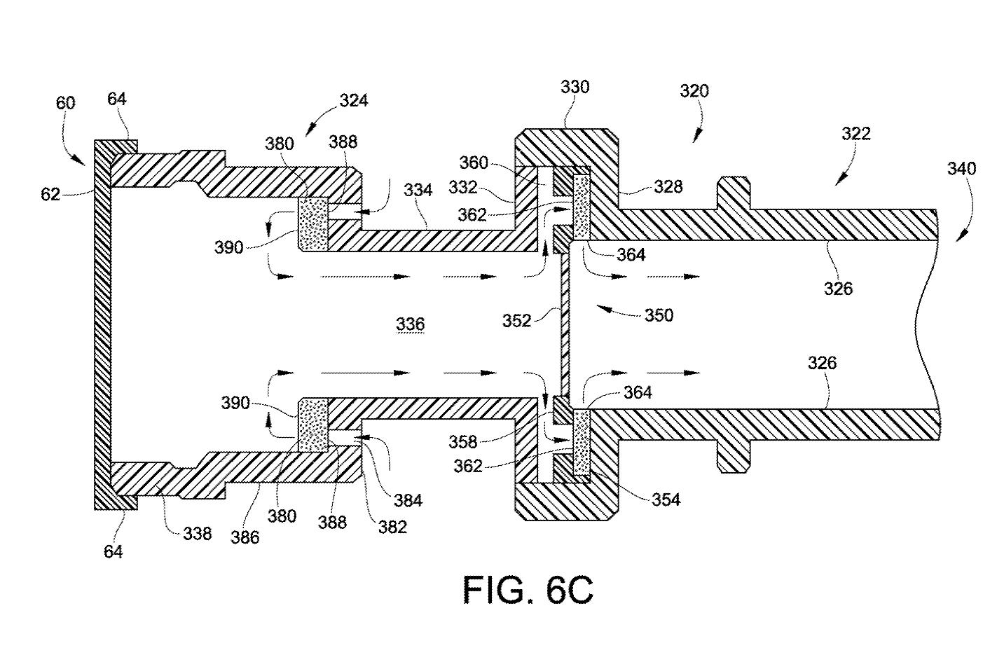

FIG. 6A illustrates an embodiment of a port 320 having a two-part body rather than the one-piece body 22, 122 and 222 of the previous embodiments. The two-part body includes a tube port body 322 and a sleeve port body 324 fixed together to form port 320. Port 320 has a first port end 338 configured to both retain a cap 60 and receive a cassette spike after cap 60 removal and a second port end 340 connected to tube 18. Tube port body 322 and sleeve port body 324 can be made of the same materials such as, for example PP, SEBS, SBR, HSBR, SSBR, PB, and combinations thereof.

Tube port body 322 includes a tube port wall 326 leading to second port end 340, a center step portion 328 extending radially outwardly from tube port wall 326, and a proximal wall 330 axially extending from the center step portion such that the proximal wall 330 has a diameter greater than tube port wall 326.

Cap 60 is fixed to first port end 338, and has substantially the same configuration as cap 60 illustrated in FIG. 2A, including cap face 62, cap flange 64 and second micronfilter 66. Sleeve port body 324 also includes a narrowed wall 334 sized to friction fit with at least a portion of a cassette spike passing through a spike path 336. Spike path 336 is defined by narrowed wall 334 and step 332 extending radially outwardly from the end of narrowed wall 334. The outermost end of step 332 serves as a first connecting end that is fixed, bonded or connected to proximal wall 330, which serves as a second connecting end, to form port 320 via heat sealing, ultrasonic welding or solvent bonding.

Port 320 further includes a filter member 350 retained within the interior of port 320, which includes a first micronfilter 354 held in place between tube port body 322 and sleeve port body 324. As illustrated in FIG. 6A, first micronfilter 354 is placed on center step portion 328 of port tube body 322 and is held in place by a first sealant 356 bonding first micronfilter 354 to center step portion 328 and proximal wall 330 of tube body 322. First micronfilter 354 also connects to a circular membrane 352 of filter member 350, the membrane extending laterally within the interior of port 320. First micronfilter 354 connects a membrane 352 via a second sealant 358 formed between first micronfilter 354 and the outer edge of membrane 352, thereby retaining membrane 352 in place across spike path 336. Membrane 352 can have a diameter substantially equal to the diameter of narrow wall 334 defining spike path 336.

Port 320 provides a steam path 360 defined between center step portion 328, proximal wall 330 and step 332. To sterilize tube 18 (see FIG. 1) and the interior of port 320, steam is injected through a second micronfilter 66 of cap 60 into first port end 338. Filter member 350 prevents steam, and any other fluids or gases, from passing through filter member 350, towards second port end 340 and into tube 18. However, first micronfilter 354 provided on center step portion 328 of tube port body 322 allows steam to pass through filter member 350 by flowing around membrane 352 and through to tube 18 by way of steam path 360 and first micronfilter 354. By providing first micronfilter 354 and steam chamber path 360, filter member 350 allows steam to sterilize both port 320 interior and tube 18 while sealing the interior and tube 18 from contaminants.

Steam enters first micronfilter 354 via a micronfilter front face 362 and exits through a micronfilter side face 364. To facilitate a side passage of steam, first micronfilter 354 is thicker than the above micronfilters (e.g. at least 1 mm thick), thus providing side face 364 having sufficient surface area to allow steam to exit side face 364 at substantially the same rate as steam enters front face 362. The thicker dimensions needed to provide side exit face 364 for first micronfilter 354 can be obtained by making first micronfilter 354 a sintered filter, such as a polyethylene (“PE”) sintered filter, which is combined with a polymer, such as polytetrafluoroethylene (“PTFE”) that is bonded or laminated to a high-density polyethylene (“HDPE”) backing. The sintered portion of micronfilter 354 provides greater pore size ranges, such as between 7 and 150 microns, thus providing the necessary side exit face pore requirements for an exit path for steam through first micronfilter 354.

Alternatively, first micronfilter 354 can have a thinner configuration (e.g. approximately 150 microns thick) as illustrated in FIG. 6B, such that the side face of the micronfilter lacks sufficient surface area for steam to exit. With thin micronfilter 354, steam enters again through face 362. To facilitate passage of steam, a bore 370 is formed from a micronfilter rear face 366 through center step 328 to a steam outlet 372 on tube port wall 326. A portion of rear face 366 substantially equal in diameter to the exposed portion of front face 362 is opened, allowing steam to pass through micronfilter 354 and around membrane 352. Micronfilters of this type generally include a polymer base, such as PTFE, that is bonded or laminated to a HDPE backing that assists in facilitating heat bonding of micronfilter 354 to tube body 322.

As an alternative to the previous embodiments that each employ a cap having a second micronfilter, the port itself may include the second micronfilter, removing the need for a cap with a micronfilter, simplifying cap 60. FIG. 6C illustrates a second micronfilter 380 fitted against a step portion 382 and a sleeve wall 386 of sleeve port body 324. A filter access passageway 384 through step 382 allows injected steam to enter micronfilter 380 through an entry face 388 and exit into the interior of port 320 through an exit face 390.

It should be understood that a one-piece port design, such as the embodiments illustrated in FIGS. 2 to 4 for example, could also provide a second micronfilter on the port itself rather than on a cap. In the case of the one-piece port 20 of FIG. 2A, for example, body 22 can provide a filter access passageway through wall 24 adjacent to first port end 28 and also provide second micronfilter 66 fitted onto wall 24 adjacent to first port end 28 to cover the filter access passageway. The filter access passageway would allow injected steam to enter second micronfilter 66 through the filter access passageway and exit into the interior of port 20 through second micronfilter 66.

To sterilize tube 18 and the interior of port 320, steam is injected through second micronfilter 380 of port 320 via filter access 384. As in the case of the embodiments illustrated in FIGS. 6A and 6B, filter member 350 prevents steam, and any other fluids or gases, from passing through filter member 350, towards second port end 340 and into tube 18. However, first micronfilter 354 provided on center step portion 328 of port tube body 322 allows steam to pass through filter member 350 by flowing around membrane 352 and through to tube 18 by way of a steam path 360 and first micronfilter 354. By providing first micronfilter 354 and steam path 360, filter member 350 allows steam to sterilize both port 320 interior and tube 18 while sealing the interior and tube 18 from contaminants. Sterile port 320 is thereafter ready for connection to a dialysis cassette.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A medical connector comprising:
a body having a first end, a second end and a body wall defining an interior,
a removable cap fixed to the first end to seal the first end, the cap having a first filter;
a filter plug having a proximal end, a distal end and a second filter, the filter plug defining a spike path on an interior of the filter plug, the proximal end fluidly sealed to an inside surface of the body wall, the distal end having a pierceable membrane that blocks the spike path; and
a steam path through the body via the first and second filters.

2. The medical connector of claim 1, wherein at least a portion of the filter plug is tapered.

3. The medical connector of claim 2, wherein at least a portion of the body wall is tapered such that the tapered portion of the filter plug seals against the tapered portion of the body wall.

4. The medical connector of claim 1, wherein the filter plug and the membrane are formed from a single molding.

5. The medical connector of claim 1, wherein the second filter is positioned at the proximal end of the filter plug.

6. The medical connector of claim 1, wherein the second filter is positioned outside the spike path.

7. The medical connector of claim 1, wherein the second filter has a disc or ring shape.

8. The medical connector of claim 1, wherein the second filter includes a plurality of small filters.

9. The medical connector of claim 1, wherein the second filter is embedded into or fastened against a wall of the filter plug for passage of steam through the plug.

10. The medical connector of claim 1, wherein a dialysis cassette spike engages the first end of the body.

11. The medical connector of claim 1, wherein the second end of the body is configured to be attached to a tube.

12. A medical connector comprising:
a cap having a first filter;
a first port including an end and a first connecting end, wherein the cap is fixed to the end to seal the end;
a second port having a second connecting end, the second connecting end fixed to the first connecting end to connect the first port and second port and define a spike path through an interior of the connected first and second ports; and
a filter member including a second filter and a membrane, the second filter fitted to an interior surface of the second port, and the membrane bonded to the second filter such that the membrane blocks the spike path.

13. The medical connector of claim 12, wherein the first port and the second port, when connected via the first and second connecting ends, define a steam path though the interior of the connected first and second ports, wherein at least a portion of the steam path is exterior to the spike path.

14. The medical connector of claim 13, wherein the second filter includes a front face and a side face, both faces forming a portion of the steam path.

15. The medical connector of claim 13, wherein the second filter includes a front face and a rear face, both faces forming a portion of the steam path.

16. The medical connector of claim 15, the second port including a bore formed at the rear face of the second filter such that steam passing through the rear face passes through the bore into the interior of the connected first and second ports.

17. A medical connector comprising:
a body comprising a first end, a second end, and a wall, the body defining a spike path and the wall defining a passageway;
a first filter provided on an inner surface of the wall, blocking the passageway;
a removable cap fixed to the first end to seal the first end; and
a filter member comprising a second filter and a membrane, the second filter fitted to the inner surface of the wall, and the membrane bonded to the second filter such that the membrane blocks the spike path.

18. The medical connector of claim 17, wherein the body is formed from a first port having a first connecting end and a second port having a second connecting end, the first connecting end fixed to the second connecting end to connect the first port to the second port and form the body.

19. The medical connector of claim 18, wherein the spike path travels through the first port and the second port.

20. The medical connector of claim 18, wherein the second filter is fitted to the second connecting end.

21. The medical connector of claim 17, wherein the first filter is provided exterior to the spike path.

22. The medical connector of claim 17, wherein the second filter is provided exterior to the spike path.

23. The medical connector of claim 17, wherein the second end of the body is connected to a tube that is pre-attached to a medical container.
